(19) United States
(12) Patent Application Publication
(10) Pub. No.: US 2014/0150182 A1
(43) Pub. Date: Jun. 5, 2014

(54) PROPERTY INFORMATION ACQUIRING APPARATUS

(71) Applicant: Hiroshi Nishihara, Kawasaki-shi (JP)

(72) Inventor: Hiroshi Nishihara, Kawasaki-shi (JP)

(73) Assignee: CANON KABUSHIKI KAISHA, Tokyo (JP)

(21) Appl. No.: 14/123,272

(22) PCT Filed: Jul. 23, 2012
(86) PCT No.: PCT/JP2012/004671

§ 371 Date: Dec. 2, 2013

(30) Foreign Application Priority Data
Jul. 26, 2011 (JP) 2011-163042

(51) Int. Cl. A61B 6/04

(52) U.S. Cl. CPC A61B 6/0435
USPC 5/601; 5/621

(57) ABSTRACT

A property information acquiring apparatus according to the present invention includes a supporting unit that supports an object person, a holding unit that holds an object area, and a receiving unit that receives information about properties of the object area by being positioned near the supporting unit, wherein the holding unit includes a restricting unit that restricts deformation generated in the supporting unit by the object person being supported by the supporting unit.

### TECHNICAL FIELD

The present invention relates to an apparatus that acquires properties of an object body.

### BACKGROUND ART

A breast examination apparatus including a bed on which an object person is placed face down and in which breasts of the object person are inserted into a hole provided in the bed is known. Among such breast examination apparatuses, a breast examination apparatus in which inserted breasts are held and pressed between a breast pressing plate made of a material that allows X rays to pass through and a radiographic plate including a sensor is disclosed (see, for example, Patent Literature 1).

FIG. 5 shows a breast examination apparatus disclosed by Patent Literature 1. A fixed pressing plate 53 and a pressing plate 38 capable of being driven with respect to the pressing plate 53 are provided. In such an apparatus, an object person lets her breasts sag downward through a breast insertion opening provided in a bed as a support platform and the saggy breasts sandwiched between the breast pressing plates are irradiated with X rays to take radiographs. This is due to consideration given to be able to restrict movement of an object person and make correct measurements by constructing an apparatus capable of taking radiographs while the object person is in a relaxed state without straining the posture of the object person.

Also, a breast examination apparatus including a breast pressing plate made of a material that allows X rays and ultrasound waves to pass through to obtain radiographs and ultrasound wave echo images of breasts pressed by the breast pressing plate is disclosed (see, for example, Patent Literature 2).

Further, technology used for diagnosis by determining neovascularization involved in tumor growth or metabolism of oxygen of hemoglobin from optical absorption properties of hemoglobin contained in blood by using near infrared light at wavelengths of about 600 to 1500 nm at which transmission properties in body tissues are excellent is known. One of such technology uses the photoacoustic effect. The photoacoustic effect is a phenomenon in which when a substance is irradiated with a pulsed light in nanoseconds, the substance absorbs light energy in accordance with optical absorption properties and an elastic wave is generated by the substance being expanded instantaneously. The elastic wave is detected by an ultrasonic transducer and a received signal is obtained. By mathematically performing analysis processing of the received signal, the sound pressure distribution generated by the photoacoustic effect can be converted into images. Because hemoglobin has a high absorption factor of near infrared light compared with water, fat, and proteins constituting a body tissue, neovascularization or metabolism of oxygen can suitably be measured by the above method. Clinical research applied to diagnosis of breast cancer or the like by using such a photoacoustic effect is actively pursued.

A breast pressing plate as described above may also be provided in a breast examination apparatus using the photoacoustic effect. Objectives thereof include preventing breasts from moving during measurement to change the measuring point and also making the breasts thinner by pressing to obtain an image of deeper portions.

In a breast examination apparatus disclosed by Patent Literature 1, skins, panniculi, or muscles near the rib or clavicle (chest wall portion) not held between the pressing plate 53 and the pressing plate 38 sag to the outer side of the breast pressing plates under the influence of gravity. These sagging skins, panniculi, or muscles act as a blind of an irradiation region of X rays so that images near the chest wall cannot be obtained. Also in a breast examination apparatus using the photoacoustic effect, sagging skins, panniculi, or muscles interfere with an ultrasonic transducer so that images near the chest wall cannot be obtained. Thus, it is necessary to improve interference with measurement by skins, panniculi, or muscles near breasts (object area) being pressed out.

This problem is not limited to measurements by a bed-type (prone) apparatus and also arises in a standing apparatus described in, for example, Patent Literature 2 and improvements thereof are demanded.

### CITATION LIST

### Patent Literature

[PTL 1]
Japanese Patent No. 2691073
[PTL 2]
Japanese Patent Application National Publication (Laid-Open) No. 09-504211

### SUMMARY OF INVENTION

### Technical Problem

The present invention has been made in view of the above circumstances and an object thereof is to provide an apparatus that is capable of measuring a whole object area of an object person in a wide range and acquires property information of an object body.

### Solution to Problem

The present invention provides a property information acquiring apparatus, comprising:

a supporting unit that supports an object person and includes an aperture through which an object area of the object person is inserted;

a holding unit that holds the object area inserted through the aperture; and

a receiving unit that is arranged near the supporting unit and receives information about properties of the object area, wherein

the holding unit includes a restricting unit that restricts deformation generated in the supporting unit by the object person being supported by the supporting unit.

### Advantageous Effects of Invention

According to the present invention, a property information acquiring apparatus of an object body capable of measuring an object area of an object person can be provided.

Further features of the present invention will become apparent from the following description of exemplary embodiments with reference to the attached drawings.

### BRIEF DESCRIPTION OF DRAWINGS

FIG. 1A is a perspective view showing an outline of a breast examination apparatus.

FIG. 1B is a partial sectional view showing the outline of the breast examination apparatus.

FIG. 2A is a perspective view showing a configuration of a measuring unit.

FIG. 2B is a partial sectional view showing the configuration of the measuring unit.

FIG. 2C is a partial enlarged view showing a region A of the measuring unit.

FIG. 2D is a partial enlarged view showing a region B of the measuring unit.

FIG. 3 is a block diagram of an ultrasonic transducer unit.

FIG. 4 is another example of first and second weight supporting units.

FIG. 5 is a block diagram of a breast pressing apparatus in Background Art.

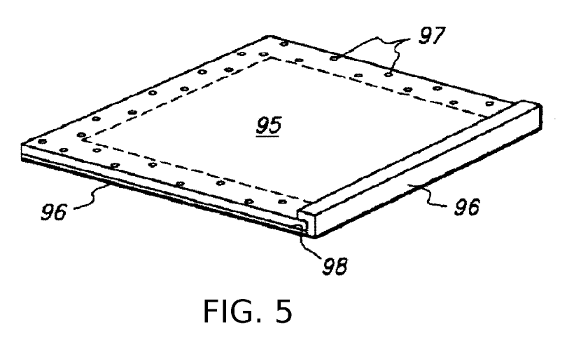

### DESCRIPTION OF EMBODIMENTS

The best mode for carrying out the present invention will be described in the following embodiment.

### Embodiment

In the embodiment, a configuration example of a breast examination apparatus using the photoacoustic effect, which is a property information acquiring apparatus to which the present invention is applied, will be described.

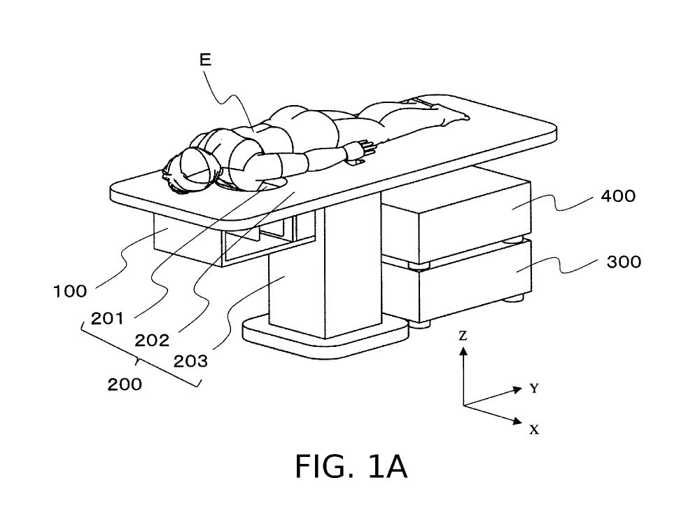

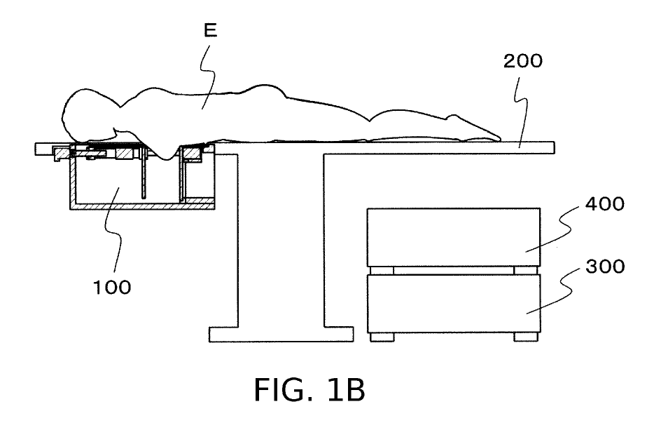

FIGS. 1A and 1B show schematic diagrams of the breast examination apparatus using the photoacoustic effect. FIG. 1A is a perspective view and FIG. 1B is a partial sectional view viewed from the X direction of FIG. 1A.

In FIGS. 1A and 1B, reference numeral 100 is a measuring unit, reference numeral 200 is a bed unit, reference numeral 300 is a light source unit, reference numeral 400 is an electric unit, and E is an object person.

The measuring unit 100 is an apparatus to measure breasts as an object area of an object person using photoacoustic effect and includes, though details thereof will be described later, a chest wall supporting plate constituting a supporting unit supporting the object person, an ultrasonic transducer as a receiving unit, and a pressing plate as a holding unit that holds the object area.

The bed unit 200 is an apparatus on which to place the object person E face down (prone position), is provided with a breast insertion opening 201 as an aperture through which breasts as an object area of the object person, and includes a bed 202 constituting a supporting unit supporting the object person together with the above chest wall supporting plate and a bed pillar 203 supporting the bed 202.

The light source unit 300 as a light irradiating unit irradiating breasts as an object area with light includes a laser light source that emits pulsed light on the order of nanoseconds of a specific wavelength shone on the breasts of the object person E. The light emitted from the laser light source is guided into the measuring unit 100 by a light guiding optical system such as an optical fiber (not shown). The wavelength of light emitted from the laser light source is selected in accordance with the absorption spectrum of water, fat, proteins, oxygenated hemoglobin, and reduced hemoglobin constituting a body tissue. As an example, because the absorption by water as a main component in a body internal tissue is small, the wavelength in the range of 600 to 1500 nm at which light is well transmitted and spectra of fat, oxygenated hemoglobin, and reduced hemoglobin are featured is appropriate. As a concrete example of the laser light source, a semiconductor laser or wavelength tunable laser generating different wavelengths may be configured.

The electric unit 400 includes a power supply unit that supplies power to the measuring unit 100 and the light source unit 300, a control unit that controls these units, and a signal processing unit that processes a signal measured by the measuring unit 100. The signal processing unit converts the sound pressure distribution of an elastic wave (acoustic wave) generated by the photoacoustic effect into images.

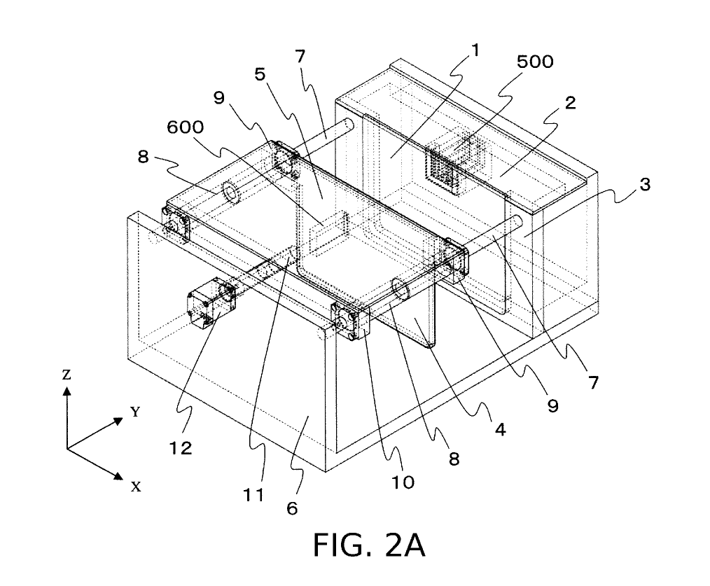

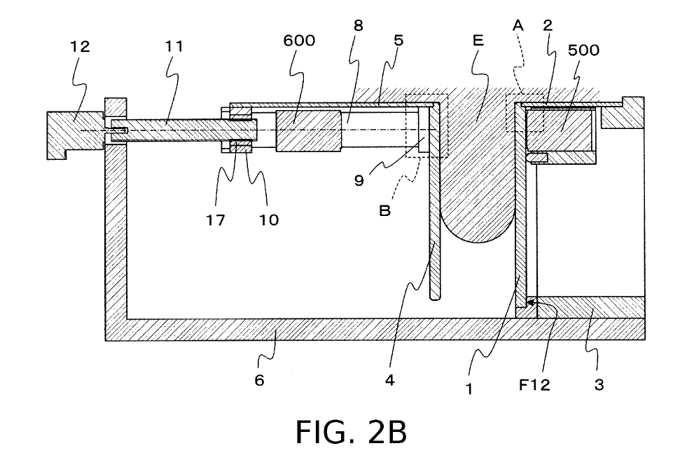

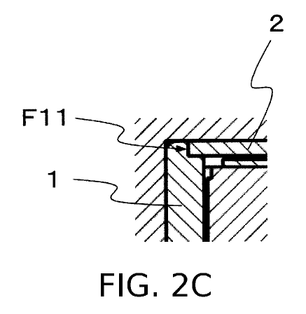

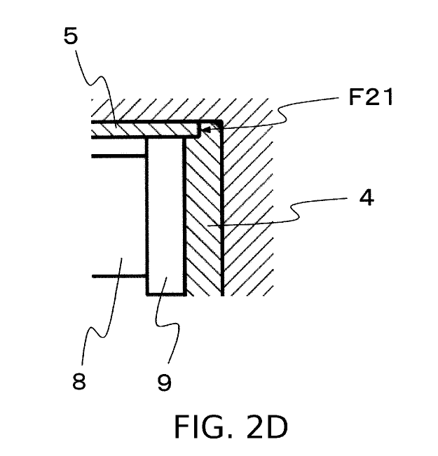

The block diagram of the measuring unit 100 is shown in FIGS. 2A to 2D. FIG. 2A is a perspective view, FIG. 2B is a partial sectional view viewed from the X direction of FIG. 2A, FIG. 2C is a partial enlarged view of a region A of FIG. 2B, and FIG. 2D is a partial enlarged view of a region B of FIG. 2B.

A first pressing plate 1 (first holding unit) that presses and holds caudal (foot-side) breasts of the object person E and a first chest wall supporting plate 2 supporting the chest wall near the underbust are mounted on a first pressing plate supporting base 3.

An ultrasonic transducer unit 500 including an ultrasonic transducer 13 (not shown) as a receiving unit to receive an acoustic wave generated by breasts as an object area after breasts as an object area being irradiated with light by the light source unit 300 as a light irradiating unit is mounted on the first pressing plate 1 in close contact therewith via a matching agent. The ultrasonic transducer unit 500 is arranged in such a way that the ultrasonic transducer 13 (not shown) as a receiving unit is positioned near the first chest wall supporting plate 2 constituting a supporting unit. Then, the ultrasonic transducer unit 500 is driven to scan in the X and Z directions (on the XZ plane) of FIG. 2A by a scanning mechanism (not shown).

A second pressing plate 4 (second holding unit) that presses and holds cephalic breasts of the object person E and a second chest wall supporting plate 5 supporting the cephalic chest wall are mounted on a slide mechanism moving in the Y direction of FIG. 2A. The slide mechanism is constituted of two principal axes 7 fixed to the first pressing plate supporting base 3 and a second pressing plate supporting base 6, a bearing 8 sliding by being guided by the principal axes 7, and a first bearing housing 9 and a second bearing housing 10 holding the bearing 8. The second bearing housing 10 is provided with a nut 17 and the second pressing plate 4 is moved in the Y direction of FIG. 2A by a screw 11 being rotated by a motor 12.

In the present embodiment, the first pressing plate 1 (first holding unit), the second pressing plate 4 (second holding unit), and the above slide mechanism constitute a press holding apparatus. Objectives of including the apparatus include preventing breasts from moving during measurement to change the measuring point and also making the breasts thinner by pressing to enable conversion of deeper portions into images. Thus, breasts are pressed and held by two pressing plates. While breasts as an object area are pressed and held in the present embodiment, pressing is not needed if it is enough to hold breasts as an object area in such a way that the measuring point thereof does not change. In such a case, the slide mechanism or the second pressing plate may become unnecessary depending on the shape or arrangement of the first pressing plate (first holding unit).

Moreover, an illumination unit 600 guiding laser light emitted from the light source unit 300 to breasts is provided. The illumination unit 600 is driven to scan in synchronization with driving of the ultrasonic transducer unit 500 in the X and Z directions (on the XZ plane) by a scanning mechanism (not shown).

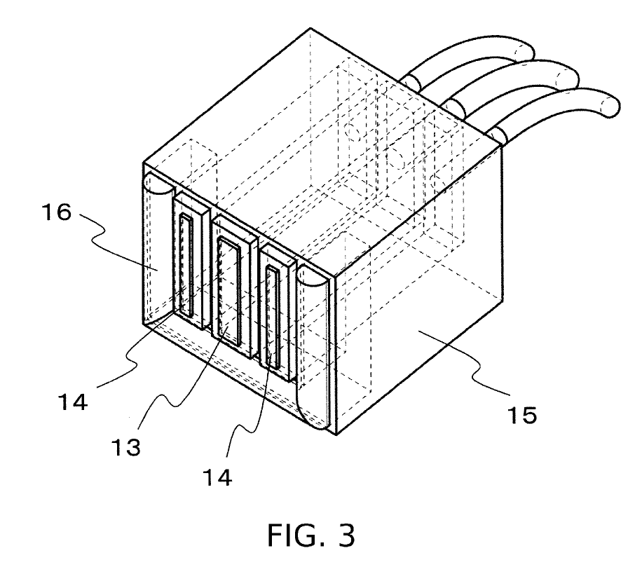

FIG. 3 is a block diagram of the ultrasonic transducer unit 500. The ultrasonic transducer unit 500 includes a housing 15 on which the ultrasonic transducer 13 as a receiving unit and an illuminating optical system 14 are mounted. The housing 15 is also provided with a seal member 16 to hold the matching agent between the first pressing plate 1 and the ultrasonic transducer 13.

Each structural element will be described in detail below.

In the present embodiment, the ultrasonic transducer 13 as a receiving unit is arranged opposite to breasts as an object area via the first pressing plate 1 as a first holding unit. Thus, the first pressing plate 1 preferably has high transmission properties and low attenuation properties with respect to an elastic wave (acoustic wave) generated by the photoacoustic effect and also has high transmission properties and low attenuation properties with respect to light emitted from a laser light source. Examples of the material forming the first pressing plate 1 include quartz glass, polymethyl pentene polymers, polycarbonate, and acryl. It is similarly necessary for the matching agent to have high transmission properties and low attenuation properties with respect to an elastic wave (acoustic wave) generated by the photoacoustic effect and also have high transmission properties and low attenuation properties with respect to light emitted from a laser light source. Examples of the matching agent include water, castor oil, gel for ultrasonic echo examination, and polyethylene glycol.

The second pressing plate 4 as a second holding unit is a flat plate having high transmission properties and low attenuation properties with respect to light emitted from a laser light source. Examples of the material forming the second pressing plate 4 include glass, polymethyl pentene polymers, polycarbonate, and acryl.

The first chest wall supporting plate 2 is provided between the chest wall of the object person E and the ultrasonic transducer unit 500. The second chest wall supporting plate 5 is provided between the chest wall of the object person E and the illumination unit 600. The first chest wall supporting plate 2 and the second chest wall supporting plate 5 are provided to support a portion (chest wall or the like) near breasts (object area) of the object person to prevent skins, panniculi, or muscles near the rib or clavicle to sag to the outer side of the breast pressing plates and radiographic plates under the influence of gravity, which is a problem of a conventional breast examination apparatus. Accordingly, interference of the chest wall, which is sagging due to gravity, with the ultrasonic transducer unit 500 or the illumination unit 600 can be avoided in a prone type property information acquiring apparatus like in the present embodiment.

The first chest wall supporting plate 2 and the second chest wall supporting plate 5 are deformed (bent or deflected) in the −Z direction of FIG. 2A by supporting the object person E. It is necessary to set the scanning range in the Z direction of the ultrasonic transducer unit 500 to a range that is not affected even if the first chest wall supporting plate 2 is bent. It is also necessary to set the scanning range in the Z direction of the illumination unit 600 to a range that is not affected even if the second chest wall supporting plate 5 is bent. Thus, making the amount of deformation (amount of bending or deflection) of the first chest wall supporting plate 2 and the second chest wall supporting plate 5 smaller leads to extending an image acquisition region near the chest wall.

Thus, in the present invention, the pressing plate 1 as a first holding unit includes a restricting unit to restrict the deformation (bending or deflection) of the first chest wall supporting plate 2 as a first supporting unit. More specifically, as shown in FIG. 2C, a portion (hereinafter, this portion will be called a first weight supporting unit W11) holding the first chest wall supporting plate 2 as a first supporting unit is included on the upper portion of the first pressing plate 1 as a first holding unit. By mounting the first chest wall supporting plate 2 on the first weight supporting unit W11, the first pressing plate 1 restricts deformation of the first chest wall supporting plate 2 to act as a reinforcing member to make the amount of deformation (amount of bending or deflection) smaller. If a screw is used to mount the first chest wall supporting plate 2 on the first weight supporting unit W11, a portion of the screw going through the first pressing plate 1 may prevent an elastic wave generated by the photoacoustic effect and light emitted from a laser light source from passing through. Thus, it is better to use an adhesive to mount these members. Alternatively, these members may be brought into contact without adhesion or may be mounted with a slight gap in such a way that the first chest wall supporting plate 2 comes into contact with the first weight supporting unit W11 when the first chest wall supporting plate 2 is bent due to the weight of the object person. Here, the first weight supporting unit W11 corresponds to a first restricting unit of the present invention.

In the present embodiment, as shown in FIG. 2B, a second weight supporting unit W12 including a surface substantially perpendicular to the direction in which the weight of the object person is applied to the upper portion of the first pressing plate supporting base 3 is provided, as a preferred embodiment, to make the amount of bending (or deflection) of the first chest wall supporting plate 2 still smaller. By mounting the first chest wall supporting plate 2 on the second weight supporting unit W12, like the above case, the first pressing plate supporting base 3 acts as a reinforcing member to make the amount of bending (or deflection) of the first chest wall supporting plate 2 smaller. Thus, by causing a portion (top surface) of the supporting base 3 on which the first pressing plate 1 and the first chest wall supporting plate 2 are mounted to function as abed and holding the first chest wall supporting plate 2 by this portion, deformation of the first chest wall supporting plate 2 can be restricted even if the chest wall supporting plate is made still thinner. As a result, as shown in FIG. 2B, an acoustic wave can be received from a wide range of the whole breasts as an object area. That is, by making the chest wall supporting plate thinner than the bed, in other words, by making only a portion of the supporting unit supporting the object person near the object area thinner than other portions, the whole breasts as an object area can be measured while maintaining strength of the whole supporting unit. The thickness of the supporting unit can be understood as a distance between the surface supporting the object person and the surface on the opposite side of the surface supporting the object person. Therefore, it is preferable to make the distance between the surface supporting the object person and the surface on the opposite side of the surface supporting the object person in a portion opposite to the receiving unit smaller than the distance between the surface supporting the object person and the surface on the opposite side of the surface supporting the object person in other portions to widen the measuring range. When mounting the first chest wall supporting plate 2 on the second weight supporting unit W12, a screw or an adhesive may be used for bonding. Alternatively, these members may be brought into contact with each other without bonding these members.

To restrict deformation (bending or deflection) of the second chest wall supporting plate 5, as shown in FIG. 2D, a first weight supporting unit W21 including a surface substantially perpendicular to the direction in which the weight of the object person is applied to the upper portion of the second pressing plate 4 is provided. By mounting the second chest wall supporting plate 5 on the first weight supporting unit W21, the second pressing plate 4 acts as a reinforcing member restricting deformation (bending or deflection) of the second chest wall supporting plate 5. For the mounting of the second chest wall supporting plate 5 on the first weight supporting unit W21, the same method as that used for mounting of the first chest wall supporting plate 2 and the first pressing plate 1 can be adopted.

To make the amount of deformation (bending or deflection) of the second chest wall supporting plate 5 still smaller, as shown in FIG. 2B, a second weight supporting unit W22 including a surface substantially perpendicular to the direction in which the weight of the object person is applied to the upper portion of the first bearing housing 9 and the second bearing housing 10 may be provided. By mounting the second chest wall supporting plate 5 on the second weight supporting unit W22, the two principal axes 7 fixed to the first pressing plate supporting base 3 and the second pressing plate supporting base 6 act as reinforcing members that make the amount of bending (or deflection) of the second chest wall supporting plate 5 smaller.

A material with a large Young's modulus is preferable as the material constituting the first chest wall supporting plate 2 and the second chest wall supporting plate 5. As an example of such a material, a metal or a metallic compound can suitably be used. For example, tungsten carbide having about twice a Young's modulus as that of iron is one of preferable materials. Stainless superior in shape workability and strength is also one of preferable materials.

As an example, the first pressing plate 1 is formed of polymethyl pentene polymers to a thickness of 10 mm, the first chest wall supporting plate 2 is formed of tungsten carbide to a thickness of 3 mm, and the first pressing plate supporting base 3 is formed of aluminum. Then, calculation shows that the bending (or deflection) when a first weight supporting unit W1 with the width of 5 mm and a second weight supporting unit W2 with the width of 10 mm are provided and a load of 800 N is applied to the upper portion of the first chest wall supporting plate 2 is about 0.12 mm.

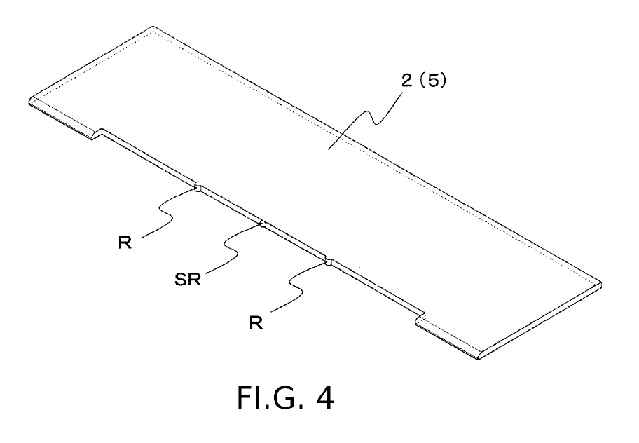

The first and second weight supporting units shown in the present embodiment are supporting units in a plane shape, but may have, as shown in FIG. 4, a spherical surface SR or a cylindrical surface R.

Aluminum, iron, stainless, or the like may be used as materials constituting the first pressing plate supporting base 3, the second pressing plate supporting base 6, the first bearing housing 9, and the second bearing housing 10. The principal axis 7 is formed of a member of a steel material in a cylindrical shape whose surface is treated to harden. The bearing 8 may be formed of a linear bush or solid bearing that can slide smoothly even if the weight of the object person E is applied. The screw 11 and the nut provided in the second bearing housing 10 may be formed of a ball screw that can be driven with a small coefficient of friction. A DC motor, an AC motor, a stepping motor or the like may be used as the motor 12.

The ultrasonic transducer 13 is formed of a piezoelectric element having a piezoelectric effect that converts a change in pressure caused by a received elastic wave into an electric signal and has a plurality of piezoelectric elements arranged in, as shown in FIG. 3, a substantial rectangular shape. Neovascularization involved in growth of tumor such as cancer is known to grow when the size of tumor is 2 to 3 mm or more. Thus, a piezoelectric ceramic material suitable for detection of an elastic wave of 0.5 MHz to several tens of MHz generated from an optical absorber of a few mm due to the photoacoustic effect and typified by PZT (lead zirconium titanate) may be used as the piezoelectric element. Also, a polymeric piezoelectric film material typified by PVDF (polyvinylidenedifluoride) may be used. The ultrasonic transducer 13 is connected to a signal processing apparatus of the electric unit 400 via a cable.

The illuminating optical system 14 is formed of a bundle of a plurality of optical fibers. The light emission end of optical fiber is formed into a substantial rectangular shape as shown in FIG. 3 by adjusting the arrangement of optical fibers. An illuminating optical system equivalent to the illuminating optical system 14 described above is used for the illumination unit 600.

In a breast examination apparatus according to the present embodiment, as described above, skins, panniculi, or muscles in a portion (chest wall or the like) near breasts as an object area are supported by the first chest wall supporting plate 2 and the second chest wall supporting plate 5. Thus, sagging skins, panniculi, or muscles will not interfere with an ultrasonic transducer. Moreover, by providing a restricting unit that restricts deformation of a chest wall supporting plate as a supporting unit in a unit (pressing plate) holding the object area, bending (or deflection) of the first chest wall supporting plate 2 and the second chest wall supporting plate 5 can be made smaller.

Accordingly, a living organism examination apparatus capable of obtaining images near the chest wall of an object person and using a photoacoustic effect can be provided.

In the above example, the present invention has been described by taking a breast examination apparatus that receives an elastic wave generated by being irradiated with light and acquires property information of an object body as an example, but the present invention is not limited to such an example and an X-ray irradiation type mammography using an X-ray irradiating unit that irradiates an object area with X rays and a receiving unit that receives X rays with which the object area is irradiated by the X-ray irradiating unit is one of forms to which the present invention can be applied.

In the above example, the present invention has been described by taking a prone measuring apparatus as an example, but the present invention is not limited to such an example and can also be applied to a standing object body information acquiring apparatus.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2011-163042, filed on Jul. 26, 2011, and Japanese Patent Application No. 2012-153633, filed on Jul. 9, 2012, which are hereby incorporated by reference herein in their entirety.

1. A property information acquiring apparatus, comprising:
a supporting unit that supports an object person who is an examinee and includes an aperture through which an object area of the examinee is inserted;
a holding unit that holds the object area inserted through said aperture; and
a receiving unit that is arranged near said supporting unit and receives information about properties of the object area,
wherein said holding unit includes a restricting unit that restricts deformation generated in said supporting unit by the examinee being supported by said supporting unit.

2. The property information acquiring apparatus according to claim 1, wherein said restricting unit is a part of said holding unit that holds said supporting unit.

3. The property information acquiring apparatus according to claim 1, wherein said supporting unit includes a bed.

4. The property information acquiring apparatus according to claim 1, further comprising:
a light irradiating unit that irradiates the object area with light,
wherein said receiving unit receives an acoustic wave generated in the object area being irradiated with the light from said light irradiating unit.

5. The property information acquiring apparatus according to claim 4, wherein said receiving unit is arranged opposite to the object area via said holding unit and said holding unit is polymethyl pentene.

6. The property information acquiring apparatus according to claim 1, further comprising:
an X-ray irradiating unit that irradiates the object area with X rays,
wherein said receiving unit receives the X rays with which the object area is irradiated by said X-ray irradiating unit.

7. The property information acquiring apparatus according to claim 1, wherein said supporting unit is made of a metal or a metallic compound.

8. The property information acquiring apparatus according to claim 7, wherein said supporting unit is made of tungsten carbide.

9. The property information acquiring apparatus according to claim 7, wherein said supporting unit is made of stainless steel.

10. The property information acquiring apparatus according to claim 1, wherein a distance between a surface supporting the examinee and a surface on an opposite side of the surface supporting the examinee in a portion opposite to said receiving unit is smaller than the distance between said surface supporting the examinee and said surface on the opposite side of said surface supporting the examinee in other portions.
